(19) United States
(12) Patent Application Publication
(10) Pub. No.: US 2023/0363335 A1
(43) Pub. Date: Nov. 16, 2023

(54) STANDARDIZED AND VIRUS-FREE RAPID PROPAGATION METHOD OF DIANTHUS CARYOPHYLLUS

(71) Applicant: Flower Research Institute, Yunnan Academy of Agricultural Sciences, Kunming (CN)

(72) Inventors: Yiping ZHANG, Kunming (CN); Yunhui QU, Kunming (CN); Yan SU, Kunming (CN); Yalian JIANG, Kunming (CN); Feng XU, Kunming (CN); Jihua WANG, Kunming (CN); Suping QU, Kunming (CN); Lihua WANG, Kunming (CN); Lifang ZHANG, Kunming (CN); Xiumei YANG, Kunming (CN); Axiang ZHAO, Kunming (CN)

(73) Assignee: Flower Research Institute, Yunnan Academy of Agricultural Sciences, Kunming (CN)

(21) Appl. No.: 17/844,766

(22) Filed: Jun. 21, 2022

(30) Foreign Application Priority Data
May 10, 2022 (CN) 202210504045.7

(51) Int. Cl. A01H 4/00

(52) U.S. Cl. CPC A01H 4/005; A01H 4/008

(57) ABSTRACT

The present disclosure provides a standardized and virus-free rapid propagation method of Dianthus caryophyllus, belonging to the technical field of asexual propagation. The method includes the following steps: conducting virus detection on a Dianthus caryophyllus explant, and inoculating the explant into an induction medium for induction culture; after the explant germinates, cutting buds, and determining whether a virus-free treatment is required according to a virus detection result, and continuing cultivating the buds; after cultivating to obtain cluster buds, inoculating the cluster buds into an expanding propagation medium for expanding propagation, and conducting the virus detection to eliminate a virus-carrying material; conducting expanding propagation on a virus-free material to a certain base, and transferring the virus-free material to a rooting medium for rooting culture.

### CROSS REFERENCE TO RELATED APPLICATION

This patent application claims the benefit and priority of Chinese Patent Application No. 202210504045.7, filed on May 10, 2022, the disclosure of which is incorporated by reference herein in its entirety as part of the present application.

### TECHNICAL FIELD

The present disclosure belongs to the technical field of asexual reproduction, and in particular relates to a standardized and virus-free rapid propagation method of Dianthus caryophyllus.

### BACKGROUND ART

Dianthus caryophyllus L., belonging to Dianthus, Caryophyllaceae, is one of the cut flower varieties with the largest production area and the highest quantity of sale in the world. Dianthus caryophyllus reproduction includes sexual reproduction and asexual reproduction. The sexual reproduction is generally not adopted in production due to a low seed setting rate and large variation in traits of seed progeny. At present, the asexual reproduction mainly includes tissue culture and cutting propagation. The tissue culture has high reproduction coefficient, virus removal, strong growth, desirable resistance and high yield, but requires certain equipment and has a long culture period. In recent years, most Dianthus caryophyllus seedling nursery companies in the world have adopted the tissue culture to obtain female parents of a virus-free mother seed, and the seedlings are obtained by cutting propagation of materials from a stock plant. The method can obtain a large number of high-quality sterile seedlings while reducing a production cost.

The Dianthus caryophyllus generally has symptoms due to the influence of virus diseases, such as different degrees of plant dwarfing, deformity, mosaic leaf, and necrosis, as well as smaller flower, cracking, and flower discoloration, resulting in constantly declining yield and quality of the Dianthus caryophyllus. Viruses that endanger the Dianthus caryophyllus include Carnation mottle virus (CarMV), Carnation latent virus (CLV), Carnation etched ring virus (CERV), Carnation necrotic fleck virus (CNFV), and Carnation vein mottle virus (CaVMV). Kunming, as a main producing area of the Dianthus caryophyllus in China, is seriously damaged by the CarMV and CNFV.

Therefore, it is of special significance to provide a standardized and virus-free rapid propagation method of Dianthus caryophyllus, and to establish a complete system of virus-free rapid propagation for the Dianthus caryophyllus, thereby obtaining a large number of female parent of virus-free mother seeds, providing sterile seedlings, reducing production costs, and promoting development of Dianthus caryophyllus industry.

### SUMMARY

In view of this, an objective of the present disclosure is to provide a standardized and virus-free rapid propagation method of Dianthus caryophyllus. The method establishes a complete breeding system of a virus-free mother seed of the Dianthus caryophyllus. By conducting the virus detection at specific time points of the induction culture and the expanding propagation, the method ensures that the virus-free mother seed of the Dianthus caryophyllus with a high induction rate and a desirable reproduction coefficient are cultivated.

To achieve the above objective, the present disclosure provides the following technical solutions.

The present disclosure provides a standardized and virus-free rapid propagation method of Dianthus caryophyllus, including the following steps: conducting virus detection on a Dianthus caryophyllus explant, and inoculating the explant into an induction medium for induction culture; after the explant germinates, cutting buds, and conducting virus-free treatment on virus-carrying buds according to a virus detection result, and continuing cultivating the buds; after cultivating to obtain cluster buds, inoculating the cluster buds into an expanding propagation medium for expanding propagation, and conducting the virus detection to eliminate a virus-carrying material; conducting expanding propagation on a virus-free material to a certain base, and transferring the virus-free material to a rooting medium for rooting culture.

Preferably, the induction medium and the expanding propagation medium each may be based on a Murashige and Skoog (MS) medium at a pH value of 5.8 to 6.0, with 0.5 mg/L to 1.0 mg/L of 6-benzylaminopurine (BA) and 0.1 mg/L to 0.3 mg/L of 1-naphthalene acetic acid (NAA).

Preferably, the induction culture and the expanding propagation each may be conducted at 22° C. ± 1° C. and an illumination intensity of 2,000 Lux to 4,000 Lux with an illumination time of 10 h/d to 12 h/d.

Preferably, the buds may be cut after the explant germinates and the buds grow to 2 cm to 3 cm.

Preferably, the virus detection may include the following steps:

detecting Carnation mottle virus (CarMV) by a double-antibody sandwich enzyme-linked immunosorbent assay (ELISA), and detecting Carnation necrotic fleck virus (CNFV) by an indirect ELISA; or detecting the CarMV and the CNFV by a reverse transcription-polymerase chain reaction (RT-PCR).

Preferably, the virus-free treatment may be conducted by a high-temperature treatment combined with a method for shoot apex peeling, including: treating the cut buds for 30 d to 35 d at 38° C. ± 1° C. and an illumination intensity of 1,200 Lux to 1,500 Lux with an illumination time of 14 h/d to 16 h/d, peeling the buds to a growth point containing 1 to 2 pairs of leaf primordia, and conducting cultivation on the induction medium.

Preferably, after one time of the expanding propagation, sampling may be conduct for the virus detection; after 20 d to 30 d of the expanding propagation, transferring may be conducted one time, and the virus detection may be conducted after 1 to 2 generations; and the virus detection may be repeated 2 to 3 times, and the expanding propagation may be continued after confirming that there is no virus.

Preferably, the virus-free material may be eliminated after 10 to 15 generations of the expanding propagation, the virus detection may be conducted irregularly during the period, and virus-free culture may be repeated.

Preferably, the rooting medium may be based on an MS medium at a pH value of 5.8 to 6.0, with 0.1 mg/L to 0.3 mg/L of NAA.

Preferably, the method may further include the following steps: after the rooting culture, conducting domestication on seedlings, and transplanting; 2 to 3 months after transplanting and surviving, conducting test cultivation of variety characteristics; and using a variety after confirming that there is no variation in the variety as a subsequent propagation material.

Compared with the prior art, the present disclosure has the following beneficial effects.

The present disclosure provides a standardized and virus-free rapid propagation method of Dianthus caryophyllus. The method standardizes a breeding technology of the mother seed of Dianthus caryophyllus, and conducts virus detection on the Dianthus caryophyllus at specific time points in an induction stage and an expanding propagation stage. The method ensures the cultivation of virus-free mother seed of the Dianthus caryophyllus with high induction rate and desirable reproduction coefficient, thereby obtaining a large number of female parent of virus-free mother seeds, providing sterile seedlings, reducing production costs, and promoting development of Dianthus caryophyllus industry.

### DETAILED DESCRIPTION OF THE EMBODIMENTS

The present disclosure provides a standardized and virus-free rapid propagation method of Dianthus caryophyllus. The method overcomes adverse effects of virus diseases on asexual reproduction of the Dianthus caryophyllus, to obtain a large amount of virus-free mother seeds of the Dianthus caryophyllus. The method includes the following steps: conducting virus detection on a Dianthus caryophyllus explant, and inoculating the explant into an induction medium for induction culture; after the explant germinates, cutting buds, and conducting virus-free treatment on virus-carrying buds according to a virus detection result, and continuing cultivating the buds; after cultivating to obtain cluster buds, inoculating the cluster buds into an expanding propagation medium for expanding propagation, and conducting the virus detection to eliminate a virus-carrying material; conducting expanding propagation on a virus-free material to a certain base, and transferring the virus-free material to a rooting medium for rooting culture.

In the present disclosure, Dianthus caryophyllus varieties as an asexual reproduction material include but are not limited to Hongfu, Pink Diamond, Bumblebee, and Spring Grass.

In the present disclosure, the explant is preferably lateral buds of the Dianthus caryophyllus, with a length of not greater than 12 cm; more preferably, in a female parent garden of the Dianthus caryophyllus, plants with robust growth, excellent traits, pure flower color, desirable flower shape, and no pests and diseases are selected to collect the explant, and the lateral buds with a strong base are selected. The buds longer than 12 cm, and the middle and upper buds beginning to differentiate into flower buds cannot be used; otherwise test tube seedlings may bloom.

In the present disclosure, the explant is preferably cleaned up in time after collecting, leaves are peeled off layer by layer, be careful not to tear the epidermis, and remain 2 to 3 pairs of leaves that are not stretched at a top; a stem section with the top is cut off, fully washed in clean water with washing powder, and rinsed with clean water; and the material is sterilized in a 0.1% to 0.2% mercuric chloride solution on an ultra-clean workbench for 10 min to 15 min. To prevent incomplete sterilization, the material is transferred to a 2% to 3% sodium hypochlorite solution and sterilized for 10 min, and rinsed in sterile water for 3 to 5 times. Shaking is conducted continuously during each sterilization and rinsing procedure, such that the agents are in full contact with the explant to achieve complete sterilization.

In the present disclosure, two seriously harmful CarMV and CNFV in the explant are preferably detected by ELISA or RT-PCR; more preferably, the CarMV is detected by double-antibody sandwich ELISA, and the CNFV is detected by indirect ELISA; further more preferably, when being not detected by ELISA, the virus is detected by RT-PCR.

In the present disclosure, antiserum of Dianthus caryophyllus virus is purchased from Agdia in the United States.

In the present disclosure, the sterilized explant is preferably inoculated into an induction medium for induction culture. The induction medium is preferably based on an MS medium, with 0.5 mg/L to 1.0 mg/L of BA and 0.1 mg/L to 0.3 mg/L of NAA, at a pH value of 5.8 to 6.0; more preferably, the induction medium is based on the MS medium, with 1.0 mg/L of the BA, 0.1 mg/L of the NAA, 6.0 g/L of A agar, and 3% sucrose. This can significantly improve the induction rate of Dianthus caryophyllus explant.

In the present disclosure, the induction culture is preferably conducted at 22° C. ± 1° C. and an illumination intensity of 2,000 Lux to 4,000 Lux with an illumination time of 10 h/d to 12 h/d.

In the present disclosure, after the explant germinates, buds are cut, whether to conduct a virus-free treatment is determined according to a virus detection result, and the buds are continued for cultivating; more preferably, the buds are cut after growing to 2 cm to 3 cm. In the present disclosure, the virus-free treatment is preferably conducted by a high-temperature treatment combined with a method for shoot apex peeling, including: treating the cut buds for 30 d to 35 d at 38° C. ± 1° C. and an illumination intensity of 1,200 Lux to 1,500 Lux with an illumination time of 14 h/d to 16 h/d, peeling the buds to a growth point containing 1 to 2 pairs of leaf primordia, and conducting cultivation on the induction medium. More preferably, the growth point of the peeled shoot apex has a length of 0.2 mm to 0.4 mm, further more preferably 0.2 mm.

In the present disclosure, after culturing the virus-free explant in the induction medium for 2 months to 3 months, new buds are differentiated, and after being grown to cluster buds, the buds are inoculated into an expanding propagation medium for expanding propagation and proliferation. The expanding propagation medium is preferably based on an MS medium, with 0.5 mg/L to 1.0 mg/L of BA and 0.1 mg/L to 0.3 mg/L of NAA, at a pH value of 5.8 to 6.0; more preferably, the expanding propagation medium is based on the MS medium, with 0.5 mg/L of the BA, 0.1 mg/L of the NAA, 6.0 g/L of A agar, and 3% sucrose. This can significantly improve the reproduction coefficient of Dianthus caryophyllus explant.

In the present disclosure, the expanding propagation is preferably conducted at 22° C. ± 1° C. and an illumination intensity of 2,000 Lux to 4,000 Lux with an illumination time of 10 h/d to 12 h/d.

In the present disclosure, the virus detection is conducted during expanding propagation; preferably, after one time of the expanding propagation, sampling may be conduct for the virus detection; after 20 d to 30 d of the expanding propagation, transferring may be conducted one time, and the virus detection may be conducted after 1 to 2 generations; and the virus detection may be repeated 2 to 3 times, and the expanding propagation may be continued after confirming that there is no virus; all clones with virus detected are eliminated. More preferably, the virus-free material is eliminated after 10 to 15 generations of the expanding propagation, the virus detection is conducted irregularly during the period, and virus-free culture is repeated.

According to the actual needs of production, the rooting culture is conducted after expanding propagation to a certain base number, and the virus-free propagation material is inoculated into the rooting medium for rooting culture. The rooting medium is preferably based on an MS medium, with 0.1 mg/L to 0.3 mg/L of NAA, at a pH value of 5.8 to 6.0; more preferably, the rooting medium is based on the MS medium, with 0.2 mg/L of the NAA, 6.0 g/L of A agar, and 3% sucrose. This can significantly improve the rooting rate of Dianthus caryophyllus explant.

In the present disclosure, the rooting culture is preferably conducted at 22° C. ± 1° C. and an illumination intensity of 2,000 Lux to 4,000 Lux with an illumination time of 10 h/d to 12 h/d.

In the present disclosure, the method further includes preferably the following steps: after the rooting culture, conducting domestication on seedlings, and transplanting; 2 to 3 months after transplanting and surviving, conducting test cultivation of variety characteristics; and using a variety after confirming that there is no variation in the variety as a subsequent propagation material.

In the present disclosure, by adjusting a medium formula, the induction rate, reproduction multiple and rooting rate are improved for asexual reproduction of the Dianthus caryophyllus; by the virus detection at specific time points in the induction stage and expanding propagation stage, and the virus-free treatment through high-temperature treatment combined with the method for shoot apex peeling, a large number of virus-free mother seeds of the Dianthus caryophyllus are effectively obtained. The virus-free seedlings are grown in a same greenhouse and show very well growth under routine management; the virus-free seedlings and the virus-carrying seedlings are subjected to mixed planting, and the virus-free seedlings become susceptible after 1 year, but have cut flower yield and high-quality flower rate after 3 years still higher than those of the virus-carrying seedlings. If all virus-free seedlings are planted in a same greenhouse, and attention is paid to field hygiene management, to avoid and reduce the transmission of viruses in various cultivation management processes, even in the case of non-isolated cultivation, the Dianthus caryophyllus plants that have been planted for 2 to 3 years can still reflect the high quality and high yield of virus-free seedlings obtained from the stem apex.

The technical solution provided by the present disclosure will be described in detail below with reference to examples, but they should not be construed as limiting the protection scope of the present disclosure.

### Example 1

A standardized and virus-free rapid propagation method of Dianthus caryophyllus included the following steps:
(1) Materials: in a female parent garden of the Dianthus caryophyllus, plants with robust growth, excellent traits, pure flower color, desirable flower shape, and no pests and diseases were selected to collect the explant, and lateral buds with a strong base were selected, and a length of the bud was generally not exceed 12 cm.
(2) Sterilization of explants: the explant was cleaned up in time after collecting, leaves were peeled off layer by layer, be careful not to tear the epidermis, and remain 2 pairs of leaves that were not stretched at a top; a stem section with the top was cut off, fully washed in clean water with washing powder, and rinsed with clean water; and the material was sterilized in a 0.1% mercuric chloride solution on an ultra-clean workbench for 15 min; to prevent incomplete sterilization, the material was transferred to a 2% sodium hypochlorite solution and sterilized for 10 min, and rinsed in sterile water for 3 times.
(3) Virus detection: CarMV was detected by a double-antibody sandwich ELISA, and CNFV was detected by an indirect ELISA; when being not detected by ELISA, the virus was detected by RT-PCR.

Double-antibody sandwich ELISA included: 100 µL of an antibody diluted to a working concentration with a coating buffer was added to wells of a microtiter plate, the microtiter plate was placed in a wet box, and incubated in a refrigerator at 4° C. overnight, or incubated at room temperature (20° C. to 25° C.) for 4 h. The coating buffer was a 0.05 mol/L Na₂CO₃—NaHCO₃ buffer, at a pH value of 9.6. Dianthus caryophyllus leaves and a grinding buffer were put in a disposable ziplock bag at a ratio of 1:10 (W/V), the bag was sealed tightly, and the sample was ground. The grinding buffer was obtained by: dissolving 2.0 g of a skim milk powder, 10.0 g of polyvinylpyrrolidone (PVP, MW_{24,000-40,000}), 1.3 g of anhydrous sodium sulfite, 0.2 g of NaN₃, and 10.0 g of Tween-20 in a washing buffer (PBST), diluting to 1,000 mL, and storing at 4° C. The washing buffer (PBST) was obtained by: dissolving 8.0 g of NaCl, 0.2 g of KH₂PO₄, 2.9 g of Na₂HPO₄·12H₂O, 0.2 g of KCl, and 0.5 mL of Tween-20 in distilled water, diluting to 1,000 mL, and adjusting to a pH value of 7.4. After incubation, the microtiter plate was washed 4 to 6 times with the washing buffer. 400 µL of the PBST washing buffer was added to each well and held for 1 min to 2 min each time. 100 µL of a supernatant after grinding was added to each well, and each sample to be tested was repeated twice, and a negative control and a positive control were set at the same time. The microtiter plate was placed in a wet box, incubated in a refrigerator at 4° C. overnight or incubated at 21° C. to 24° C. for 2.5 h. The plate was washed. An enzyme-labeled antibody was prepared 10 min before coating, a dilution buffer of the specific enzyme-labeled antibody was diluted to a working concentration, and 100 µL was added to each well. The microtiter plate was placed in a wet box, incubated at 21° C. to 24° C. for 2.5 h. The plate was washed. The dilution buffer of the enzyme-labeled antibody was obtained by: dissolving 2.0 g of bovine serum albumin (BSA), 10.0 g of PVP (MW_{24,000-40,000}), and 0.2 g of NaN₃ in the PBST, diluting to 1,000 mL, and storing at 4° C. 100 µL of a substrate solution was added to each well. The substrate solution was prepared for immediate use by: dissolving 1 mg of PNPP in 1 mL of a substrate buffer, with a concentration of 1 mg/mL. The substrate buffer is obtained by: dissolving 97.0 mL of diethanolamine, 0.1 g of magnesium chloride, and 0.2 g of NaN₃ in 800 mL of distilled water, adjusting to a pH value of 9.8 with 2 mol/L hydrochloric acid, diluting to 1000 mL, and storing at 4° C. The microtiter plate was placed in a wet box, to conduct color development at 21° C. to 24° C. for 30 min to 60 min in the dark. 50 µL of a stop solution was added to each well to stop the reaction. The stop solution was a sodium hydroxide solution of 3 mol/L. Within 20 min of stopping the reaction, the microtiter plate was placed in a microplate reader, an absorbance (OD₄₀₅) was measured at 405 nm, and a reaction result was recorded. If an average OD value of the sample was greater than 2 times an OD value of the negative control, it was determined to be the CarMV.

The indirect ELISA included: Dianthus caryophyllus leaves and an indirect sample extraction buffer were placed in a disposable ziplock bag at a ratio of 1:100 (W/V), the bag was sealed tightly, and the sample was ground. The sample extraction buffer was obtained by: dissolving 20.1 g of PVP (MW_{24,000-40,000}), 0.2 g of NaN₃, 3.0 g of NaHCO₃, and 1.6 g of Na₂CO₃ in distilled water, diluting to 100 mL, and storing at 4° C. 100 µL of a supernatant after grinding was added to each well, and each sample to be tested was repeated twice, and a negative control and a positive control were set at the same time. The microtiter plate was placed in a wet box and incubated at room temperature for 1 h. After incubation, the microtiter plate was washed 8 times with the PBST washing buffer. 400 µL of the PBST washing buffer was added to each well and held for 1 min to 2 min each time. After washing, the plate was dried. 100 µL of the antibody diluted to a working concentration in an ECI buffer was added to wells of the microtiter plate. The microtiter plate was placed in a wet box and incubated at room temperature for 2 h. After incubation, the microtiter plate was washed 8 times with the PBST washing buffer. 400 µL of the PBST washing buffer was added to each well and held for 1 min to 2 min each time. After washing, the plate was dried. The ECI buffer solution was obtained by: dissolving 1.0 g of BSA, 0.3 g of polyethylene glycol sorbitan monolaurate, 10.0 g of PVP (MW_{24,000-40,000}), 0.1 g of KCl, 0.1 g of K₃PO₄, 0.02 g of NaN₃, 4.0 g of NaCl, and 0.6 g of Na₃PO₄ in distilled water, diluting to 100 mL, and storing at 4° C. An enzyme-labeled antibody was prepared 10 min before coating; 100 µL of the enzyme-labeled antibody diluted to a working concentration in the ECI buffer was added to wells of the microtiter plate. The microtiter plate was placed in a wet box and incubated at room temperature for 1 h. After incubation, the microtiter plate was washed 8 times with the PBST washing buffer.400 µL of the PBST washing buffer was added to each well and held for 1 min to 2 min each time. After washing, the plate was dried.100 µL of a PNP substrate solution was added to each well. The PNP substrate solution was prepared for immediate use by: dissolving 1 mg of a PNP substrate in 1 mL of a PNP substrate buffer, with a concentration of 1 mg/mL. The PNP substrate buffer was obtained by: dissolving 40.0 mL of diethanolamine, 12.0 g of ethanolamine hydrochloride, 0.05 g of magnesium chloride hexahydrate, and 0.1 g of NaN₃ in 80 mL of distilled water, adjusting a pH value to 9.8 with 2 mol/L hydrochloric acid, diluting to 100 mL and storing at 4° C. The microtiter plate was placed in a wet box, to conduct color development for 1 h at room temperature in the dark. The microtiter plate was placed in a microplate reader, an absorbance (OD₄₀₅) was measured at 405 nm, and a reaction result was recorded. If an average OD value of the sample was greater than 2 times an OD value of the negative control, it was determined to be the CNFV.

RT-PCR included: 0.05 g of a sample tissue was added into a sterilized mortar, 1 mL of a Trizol solution was added and the sample tissue was ground into a homogenate, the homogenate was transferred into a 1.5 mL centrifuge tube, and allowed to stand at room temperature for 5 min to fully lyse the tissue. 200 µL of chloroform was added, the centrifuge tube was inverted vigorously to mix, allowed to stand for 5 min, and centrifugation was conducted at 12,000 r/min for 10 min. An upper aqueous phase was aspirated and transferred to another 1.5 mL centrifuge tube. 500 µL of isopropanol was added and mixed well, and stood at room temperature for 10 min. Centrifugation was conducted at 12,000 r/min for 5 min, a supernatant was discarded. 1 mL of 75% ethanol was added, the tube was shaken for a while, centrifuged at 7,500 r/min for 5 min, and a supernatant was carefully discarded. An RNA precipitate was just dried by standing for 5 min to 15 min at room temperature, 50 µL of double-distilled water was added to dissolve the RNA precipitate for later use. The following reagents were added in sequence to a 0.5 µL PCR tube for reverse transcription: 8 µL of ddH₂O, 0.5 µL of a 20 pmol/µL random primer, 2 µL of an RNA template, denatured at 70° C. for 10 min, and treated on ice for 5 min. 4 µL of a 5× M-MLV Reverse Transcription Buffer, 3 µL of 10 mmol/L dNTP, 0.5 µL of a 40 u/µL RNA Inhibitor, and 1 µL of a 200 u/µL M-MLV Reverse Transcriptase were added. The above components were mixed well, centrifuged at a low speed, and reacted in a thermal cycler at 42° C. for 1 h. The 5× M-MLV Reverse Transcription Buffer was prepared by 250 mmol/L Tris·HCl at pH 8.3, 375 mmol/L KCl, 15 mmol/L MgCl₂, and 50 mmol/L DTT. After the reaction, the following reagents were added to the PCR tube in sequence for PCR amplification: 32.7 µL of double-distilled water, 5 µL of a 10× PCR buffer, 1 µL of a 20 pmol/µL upstream primer, 1 µL of a 20 pmol/µL downstream primer, 0.3 µL of a 5 u/µL TaqDNA polymerase, and 10 µL of a reverse transcription product. The reagents were thoroughly mixed, and PCR amplification was conducted by: 94° C. for 10 min; denaturation at 94° C. for 30 sec, annealing at 60° C. for 60 sec, and extension at 72° C. for 60 sec, conducting 35 cycles; and extension at 72° C. for 10 min. The 10× PCR buffer was prepared by 100 mmol/L Tris·HCl at pH 8.3, 500 mmol/L KCl, and 15 mmol/L MgCl₂. An amplification product detection method was as follows: 5×TBE was diluted at 1:5 to obtain a 1×TBE working solution, a 1.5% (W/V) solution was prepared with agarose, and heated to melt the agarose completely. When the gel was cooled to 50° C. to 60° C., an appropriate amount of anthocyanin was added, poured into a platform, and cooled down. The PCR product was added with a loading buffer at a ratio of 6:1, and spotted. Electrophoresis was conducted at 1-10 V/cm until a migration of bromophenol blue exceeded ½ of a length of the gel plate, and the electrophoresis was stopped. The gel plate was taken out, and observed and photographed by a gel imaging system. The 5×TBE buffer was obtained by: dissolving 54 g of Tris, 27.5 g of boric acid, 20 mL of 0.5 mol/L EDTA (pH 8.0), diluting to 1.000 mL, and storing at 4° C. The loading buffer was obtained by: dissolving 0.25% bromophenol blue in a 40% (W/V) sucrose aqueous solution, storing at 4° C.
A CarMV primer sequence used in this method included:
   5′-primer, 5′-CGGATAGTCTTGTCAACATACGG-3′ (SEQ ID NO: 1), 3′-primer, 5′-CCTTATCGTTGCTTGCCTGT-3′ (SEQ ID NO: 2);
A CNFV primer sequence included:
   5′-primer, 5′-AAGGTATCATCGGCAGACAG-3′ (SEQ ID NO: 3), 3′-primer, 5′GAAGAATCTCGTGAAGTGGC3′ (SEQ ID NO: 4).

(4) Induction culture: the sterilized explant was inoculated into an induction medium; the induction medium was an MS-based medium, with BA 1.0 mg/L+NAA 0.1 mg/L+A agar 6.0 g/L+3% sucrose, at a pH of 5.8 to 6.0; induction culture was conducted at 22±1° C. and an illumination intensity of 3,000 Lux with an illumination time of 11 h/d.
(5) Virus-free treatment: after the explant germinated and buds grew to 3 cm, the buds were cut for virus-free treatment; the virus-free treatment was conducted by a high-temperature treatment combined with a method for shoot apex peeling, including: treating the cut buds for 34 d at 38° C. ± 1° C. and an illumination intensity of 1,300 Lux with an illumination time of 15 h/d, peeling the buds to a growth point containing 1 to 2 pairs of leaf primordia, and conducting cultivation on the induction medium; a growth point length of the peeled shoot apex was 0.2 mm.
(6) Expanding propagation: after cultivating for 2 to 3 months, new buds were differentiated, and when growing to cluster buds, the new buds were inoculated into an expanding propagation medium; the expanding propagation medium was an MS-based medium, with BA 0.5 mg/L+NAA 0.1 mg/L+A agar 6.0 g/L+3% sucrose, at a pH of 5.8 to 6.0; expanding propagation was conducted at 22±1° C. and an illumination intensity of 3,000 Lux with an illumination time of 11 h/d.
(7) Secondary virus detection: the virus detection was conducted during expanding propagation; and after one time of the expanding propagation, sampling was conduct for the virus detection; after 25 d of the expanding propagation, transferring was conducted one time, and the virus detection was conducted after 1 generation; and the virus detection was repeated 3 times, and the expanding propagation was continued after confirming that there was no virus; all clones with virus detected were eliminated.
(8) Rooting culture: according to the actual needs of production, rooting culture was conducted after expanding propagation to a certain base number; the sterilized explant was inoculated into an rooting medium; the rooting medium was an MS-based medium, with NAA 0.2 mg/L+A agar 6.0 g/L+3% sucrose, at a pH of 5.8 to 6.0; the rooting culture was conducted at 22±1° C. and an illumination intensity of 3,000 Lux with an illumination time of 11 h/d.
9) Domestication and transplanting: rooted bottle seedlings were put under strong light for 4 d, the medium adhering to the roots was removed by washing, and the roots were soaked in a 10,000-time solution of a 50% chlorothalonil wettable powder for 10 sec, and then taken out; the roots were transplanted to a peat: perlite (1:1) matrix for culture, water was drenched for root fixation thoroughly; the roots were covered with a plastic film to keep moisture and covered with a layer of shading net to shade, gradually ventilated and lightly penetrated after 14 d, and watered once a week with a ⅛ MS solution to promote growth; 2 to 3 months after transplanting and surviving, test cultivation of variety characteristics was conducted; and a variety after confirming that there was no variation in the variety was used as a subsequent propagation material; meanwhile, propagation material was obtained for cutting seedlings in the female parent garden.

### Example 2

(1) Materials: in a female parent garden of the Dianthus caryophyllus, plants with robust growth, excellent traits, pure flower color, desirable flower shape, and no pests and diseases were selected to collect the explant, and lateral buds with a strong base were selected, and a length of the bud was generally not exceed 12 cm.
(2) Sterilization of explants: the explant was cleaned up in time after collecting, leaves were peeled off layer by layer, be careful not to tear the epidermis, and remain 3 pairs of leaves that were not stretched at a top; a stem section with the top was cut off, fully washed in clean water with washing powder, and rinsed with clean water; and the material was sterilized in a 0.2% mercuric chloride solution on an ultra-clean workbench for 10 min; to prevent incomplete sterilization, the material was transferred to a 3% sodium hypochlorite solution and sterilized for 10 min, and rinsed in sterile water for 5 times.
(3) Virus detection: same as that in Example 1.
(4) Induction culture: the sterilized explant was inoculated into an induction medium; the induction medium was an MS-based medium, with BA 0.5 mg/L+NAA 0.2 mg/L+A agar 6.0 g/L+3% sucrose, at a pH of 5.8 to 6.0; induction culture was conducted at 22±1° C. and an illumination intensity of 4,000 Lux with an illumination time of 10 h/d.
(5) Virus-free treatment: after the explant germinated and buds grew to 2 cm, the buds were cut for virus-free treatment; the virus-free treatment was conducted by a high-temperature treatment combined with a method for shoot apex peeling, including: treating the cut buds for 35 d at 38° C. ± 1° C. and an illumination intensity of 1,200 Lux with an illumination time of 16 h/d, peeling the buds to a growth point containing 1 to 2 pairs of leaf primordia, and conducting cultivation on the induction medium; a growth point length of the peeled shoot apex was 0.3 mm.
(6) Expanding propagation: after cultivating for 2 to 3 months, new buds were differentiated, and when growing to cluster buds, the new buds were inoculated into an expanding propagation medium; the expanding propagation medium was an MS-based medium, with BA 1.0 mg/L+NAA 0.1 mg/L+A agar 6.0 g/L+3% sucrose, at a pH of 5.8 to 6.0; expanding propagation was conducted at 22±1° C. and an illumination intensity of 4,000 Lux with an illumination time of 10 h/d.
(7) Secondary virus detection: after one time of the expanding propagation, sampling was conduct for the virus detection; after 20 d of the expanding propagation, transferring was conducted one time, and the virus detection was conducted after 1 generation; and the virus detection was repeated 2 times, and the expanding propagation was continued after confirming that there was no virus; all clones with virus detected were eliminated.
(8) Rooting culture: according to the actual needs of production, rooting culture was conducted after expanding propagation to a certain base number; the sterilized explant was inoculated into an rooting medium; the rooting medium was an MS-based medium, with NAA 0.1 mg/L+A agar 6.0 g/L+3% sucrose, at a pH of 5.8 to 6.0; the rooting culture was conducted at 22±1° C. and an illumination intensity of 4,000 Lux with an illumination time of 10 h/d.
(9) Domestication and transplanting: the same as that in Example 1.

### Example 3

(1) Materials: in a female parent garden of the Dianthus caryophyllus, plants with robust growth, excellent traits, pure flower color, desirable flower shape, and no pests and diseases were selected to collect the explant, and lateral buds with a strong base were selected, and a length of the bud was generally not exceed 12 cm.
(2) Sterilization of explants: the explant was cleaned up in time after collecting, leaves were peeled off layer by layer, be careful not to tear the epidermis, and remain 3 pairs of leaves that were not stretched at a top; a stem section with the top was cut off, fully washed in clean water with washing powder, and rinsed with clean water; and the material was sterilized in a 0.2% mercuric chloride solution on an ultra-clean workbench for 12 min; to prevent incomplete sterilization, the material was transferred to a 3% sodium hypochlorite solution and sterilized for 10 min, and rinsed in sterile water for 4 times.
(3) Virus detection: same as that in Example 1.
(4) Induction culture: the sterilized explant was inoculated into an induction medium; the induction medium was an MS-based medium, with BA 1.0 mg/L+NAA 0.3 mg/L+A agar 6.0 g/L+3% sucrose, at a pH of 5.8 to 6.0; induction culture was conducted at 22±1° C. and an illumination intensity of 2,000 Lux with an illumination time of 12 h/d.
(5) Virus-free treatment: after the explant germinated and buds grew to 3 cm, the buds were cut for virus-free treatment; the virus-free treatment was conducted by a high-temperature treatment combined with a method for shoot apex peeling, including: treating the cut buds for 30 d at 38° C. ± 1° C. and an illumination intensity of 1,500 Lux with an illumination time of 14 h/d, peeling the buds to a growth point containing 1 to 2 pairs of leaf primordia, and conducting cultivation on the induction medium; a growth point length of the peeled shoot apex was 0.4 mm.
(6) Expanding propagation: after cultivating for 2 to 3 months, new buds were differentiated, and when growing to cluster buds, the new buds were inoculated into an expanding propagation medium; the expanding propagation medium was an MS-based medium, with BA 1.0 mg/L+NAA 0.3 mg/L+A agar 6.0 g/L+3% sucrose, at a pH of 5.8 to 6.0; expanding propagation was conducted at 22±1° C. and an illumination intensity of 2,000 Lux with an illumination time of 12 h/d.
(7) Secondary virus detection: after one time of the expanding propagation, sampling was conduct for the virus detection; after 30 d of the expanding propagation, transferring was conducted one time, and the virus detection was conducted after 2 generations; and the virus detection was repeated 2 times, and the expanding propagation was continued after confirming that there was no virus; all clones with virus detected were eliminated.
(8) Rooting culture: according to the actual needs of production, rooting culture was conducted after expanding propagation to a certain base number; the sterilized explant was inoculated into an rooting medium; the rooting medium was an MS-based medium, with NAA 0.3 mg/L+A agar 6.0 g/L+3% sucrose, at a pH of 5.8 to 6.0; the rooting culture was conducted at 22±1° C. and an illumination intensity of 2,000 Lux with an illumination time of 12 h/d.
(9) Domestication and transplanting: the same as that in Example 1.

### Example 4

### Effect of Different Induction Media on the Induction Effect of Dianthus caryophyllus

Dianthus caryophyllus explant after sterilization was subjected to an induction medium screening test. The induction medium was MS+BA 0.5 mg/L to 1.0 mg/L+NAA 0.1 mg/L to 0.3 mg/L+A agar 6.0 g/L, 3% sucrose, at a pH value of 5.8 to 6.0; the induction culture was conducted at 22±1° C. and an illumination intensity of 2,000 Lux to 4,000 Lux with an illumination time of 10 h/d to 12 h/d. The screening results are shown in Table 1.

TABLE 1

Effect of different induction media on induction rate and bud growth of Dianthus caryophyllus

| S N | Treatm ent | BA (mg/L) | NAA (mg/L) | Induction rate (%) | Bud growth |
|---|---|---|---|---|---|
| 1 | A1 | 0.50 | 0.10 | 42.50 | Buds with fast elongation and general growth |
| 2 | A2 | 0.50 | 0.20 | 51.00 | Buds with fast elongation and general growth |
| 3 | A3 | 0.50 | 0.30 | 35.00 | Buds with slow elongation and poor growth |
| 4 | A4 | 1.00 | 0.10 | 92.50 | Buds with fast elongation and well growth |
| 5 | A5 | 1.00 | 0.20 | 80.00 | Buds with fast elongation and desirable growth |
| 6 | A6 | 1.00 | 0.30 | 77.50 | Buds with slow elongation and general growth |

According to Table 1, it can be seen that MS+BA 1.0 mg/L+NAA 0.1 mg/L is more conducive to the induction of Dianthus caryophyllus explant.

### Example 5

### Effect of Different Virus-Free Methods on the Virus-free Effect of Dianthus caryophyllus

After the explant germinated and buds grew to 2 cm to 3 cm, the buds were cut and subjected to virus-free culture; three virus-free methods were used, and the most suitable virus-free method was selected for Dianthus caryophyllus.
(1) Directly shoot apex peeling method: the material was placed on an ultra-clean workbench, and tender leaves were peeled off with tweezers and a scalpel under the naked eye to expose a growth cone; a base of the bud was fixed with tweezers, and the shoot apex was continued to be peeled under a dissecting microscope at 25 to 50 times; when peeling to a growth point containing 1 to 2 pairs of leaf primordia, the shoot apex was picked on a medium with the tip of a knife.
(2) High-temperature treatment combined with shoot apex peeling method: the material was placed in a KBWF720 artificial climate box, and incubated for 30 d to 35 d at 38° C. ± 1° C. every day, with illumination for 14 h to 16 h at 1,200 Lux to 1,500 Lux, and then with darkness for 8 h to 10 h; and the shoot apex was continued to be peeled under a dissecting microscope at 25 to 50 times; when peeling to a growth point containing 1 to 2 pairs of leaf primordia, the shoot apex was picked on a medium with the tip of a knife.
(3) Ribavirin combined with shoot apex peeling method: the material was inoculated on a medium containing ribavirin for culture, namely MS+BA 0.5 mg/L to 1.5 mg/L+NAA 0.1 mg/L to 0.3 mg/L+3 mg/L to 5 mg/L ribavirin+A Agar 6.0 g/L, 3% sucrose, at a pH value of 5.8 to 6.0; after culturing for 30 d to 40 d, the shoot apex was peeled off.

The peeled shoot apex was inoculated on a medium including MS+BA 0.5 mg/L to 1.0 mg/L+NAA 0.1 mg/L to 0.3 mg/L+A agar 6.0 g/L, 3% sucrose, at a pH value of 5.8 to 6.0; each shoot apex was cultured and numbered separately; the culture was conducted at 22±1° C. and an illumination intensity of 2,000 Lux to 4,000 Lux with an illumination time of 10 h/d to 12 h/d. The screening results are shown in Table 2.

TABLE 2

Effect of different virus-free methods on virus-free effect of Dianthus caryophyllus

| Virus-free method | Length of growth point of peeled shoot apex (mm) | Number of peeled shoot apex (s) | Number of regenerated plant (s) | Number of virus-free regenerated plant (s) | Regenera tion rate (%) | Virus-free rate (%) |
|---|---|---|---|---|---|---|
| Directly shoot apex peeling | 0.2 | 30 | 17 | 9 | 56.67 | 30.00 |
| | 0.4 | 30 | 20 | 7 | 66.67 | 23.33 |
| | 0.6 | 30 | 23 | 3 | 76.67 | 10.00 |
| | 0.8 | 30 | 25 | 0 | 83.33 | 0.00 |
| | 1.0 | 30 | 28 | 0 | 93.33 | 0.00 |
| High-temperature treatment combined with shoot apex peeling | 0.2 | 30 | 14 | 12 | 46.67 | 40.00 |
| | 0.4 | 30 | 21 | 10 | 70.00 | 33.33 |
| | 0.6 | 30 | 23 | 9 | 76.67 | 30.00 |
| | 0.8 | 30 | 24 | 7 | 80.00 | 23.33 |
| | 1.0 | 30 | 22 | 5 | 73.33 | 16.67 |
| Ribavirin combined with shoot apex peeling | 0.2 | 30 | 16 | 10 | 53.33 | 33.33 |
| | 0.4 | 30 | 19 | 9 | 63.33 | 30.00 |
| | 0.6 | 30 | 22 | 8 | 73.33 | 26.67 |
| | 0.8 | 30 | 26 | 6 | 86.67 | 20.00 |
| | 1.0 | 30 | 29 | 3 | 96.67 | 10.00 |

According to Table 2, it can be seen that the high-temperature treatment combined with shoot apex peeling method has an optimal virus-free effect, and the peeled growth point is controlled at 0.2 mm.

### Example 6

### Effect of Different Expanding Propagation Media on the Expanding Propagation Effect of Dianthus caryophyllus

The virus-free Dianthus caryophyllus material was cultivated for 2 to 3 months and differentiated into new buds, and after growing to cluster buds, the buds were inoculated into the expanding propagation medium, and an expanding propagation medium screening test was conducted; there were 30 bottles per treatment, and there were 3 replicates with 10 bottles in each replicate, and statistical results were obtained after 20 d. The expanding propagation medium was MS+BA 0.5 mg/L to 1.0 mg/L+NAA 0.1 mg/L to 0.3 mg/L+A agar 6.0 g/L, 3% sucrose, at a pH value of 5.8 to 6.0; the induction culture was conducted at 22±1° C. and an illumination intensity of 2,000 Lux to 4,000 Lux with an illumination time of 10 h/d to 12 h/d. The screening results are shown in Table 3.

TABLE 3

Effect of different expanding propagation media on reproduction coefficient and growth of Dianthus caryophyllus

| S N | Treatm ent | BA (mg/L) | NAA (mg/L) | Reproduction coefficient | Growth of seedlings |
|---|---|---|---|---|---|
| 1 | B1 | 0.50 | 0.10 | 5.00 | Lots of buds with well growth |
| 2 | B2 | 0.50 | 0.20 | 4.00 | Lots of buds with desirable growth |
| 3 | B3 | 0.50 | 0.30 | 2.80 | Lots of buds with general growth |
| 4 | B4 | 1.00 | 0.10 | 4.70 | Lots of buds with desirable growth |
| 5 | B5 | 1.00 | 0.20 | 3.90 | Lots of buds with general growth |
| 6 | B6 | 1.00 | 0.30 | 3.60 | Lots of buds with general growth |

According to Table 3, it can be seen that MS+BA 0.5 mg/L+NAA 0.1 mg/L is more conducive to the expanding propagation of Dianthus caryophyllus.

### Example 7

### Effect of Different Rooting Media on the Rooting Effect of Dianthus caryophyllus

After the Dianthus caryophyllus material was subjected to expanding propagation to a certain base, rooting culture was conducted; there were 30 bottles per treatment, and there were 3 replicates with 10 bottles in each replicate, and statistical results were obtained after 20 d. The virus-free propagation material was inoculated into the rooting medium including MS++NAA 0.1 mg/L to 0.3 mg/L+A agar 6.0 g/L, 3% sucrose, at a pH value of 5.8 to 6.0; the rooting culture was conducted at 22±1° C. and an illumination intensity of 2,000 Lux to 4,000 Lux with an illumination time of 10 h/d to 12 h/d. The screening results are shown in Table 4.

TABLE 4

Effect of different rooting media on rooting rate of Dianthus caryophyllus

| SN | Treatment | NAA (mg/L) | Rooting rate (%) | Root length (cm) | Number of root (s) |
|---|---|---|---|---|---|
| 1 | C1 | 0.10 | 80 | 3.5 | 10 |
| 2 | C2 | 0.20 | 91 | 3 | 8 |
| 3 | C3 | 0.30 | 75 | 4 | 7 |

According to Table 4, it can be seen that MS+NAA 0.2 mg/L is an optimal rooting medium of Dianthus caryophyllus.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A standardized and virus-free rapid propagation method of Dianthus caryophyllus, comprising the following steps:
conducting virus detection on a Dianthus caryophyllus explant, and inoculating the explant into an induction medium for induction culture; after the explant germinates, cutting buds, and conducting virus-free treatment on virus-carrying buds according to a virus detection result, and continuing cultivating the buds; after cultivating to obtain cluster buds, inoculating the cluster buds into an expanding propagation medium for expanding propagation, and conducting the virus detection to eliminate a virus-carrying material; conducting expanding propagation on a virus-free material to a certain base, and transferring the virus-free material to a rooting medium for rooting culture;
wherein after one time of the expanding propagation, sampling is conduct for the virus detection; after 20 d to 30 d of the expanding propagation, transferring is conducted one time, and the virus detection is conducted after 1 to 2 generations, and the virus detection is repeated 2 to 3 times, and the expanding propagation is continued after confirming that there is no virus; and
wherein the virus-free material is eliminated after 10 to 15 generations of the expanding propagation, the virus detection is conducted irregularly during the period, and virus-free culture is repeated.

2. The standardized and virus-free rapid propagation method of Dianthus caryophyllus according to claim 1, wherein the induction medium and the expanding propagation medium each are based on a Murashige and Skoog (MS) medium at a pH value of 5.8 to 6.0, added with 0.5 mg/L to 1.0 mg/L of 6-benzylaminopurine (BA) and 0.1 mg/L to 0.3 mg/L of 1-naphthalene acetic acid (NAA).

3. The standardized and virus-free rapid propagation method of Dianthus caryophyllus according to claim 1, wherein the induction culture and the expanding propagation each are conducted at 22° C. ± 1° C. and an illumination intensity of 2,000 Lux to 4,000 Lux with an illumination time of 10 h/d to 12 h/d.

4. The standardized and virus-free rapid propagation method of Dianthus caryophyllus according to claim 1, wherein the buds are cut after the explant germinates and the buds grow to 2 cm to 3 cm.

5. The standardized and virus-free rapid propagation method of Dianthus caryophyllus according to claim 1, wherein the virus detection comprises the following steps:
detecting Carnation mottle virus (CarMV) by a double-antibody sandwich enzyme-linked immunosorbent assay (ELISA), and detecting Carnation necrotic fleck virus (CNFV) by an indirect ELISA; or
detecting the CarMV and the CNFV by a reverse transcription-polymerase chain reaction (RT-PCR).

6. The standardized and virus-free rapid propagation method of Dianthus caryophyllus according to claim 1, wherein the virus-free treatment is conducted by a high-temperature treatment combined with a method for shoot apex peeling, comprising: treating the cut buds for 30 d to 35 d at 38° C. ± 1° C. and an illumination intensity of 1,200 Lux to 1,500 Lux with an illumination time of 14 h/d to 16 h/d, peeling the buds to a growth point containing 1 to 2 pairs of leaf primordia, and conducting cultivation on the induction medium.

7. (canceled)

8. (canceled)

9. The standardized and virus-free rapid propagation method of Dianthus caryophyllus according to claim 1, wherein the rooting medium is based on an MS medium at a pH value of 5.8 to 6.0, with 0.1 mg/L to 0.3 mg/L of NAA.

10. The standardized and virus-free rapid propagation method of Dianthus caryophyllus according to claim 1, further comprising the following steps: after the rooting culture, conducting domestication on seedlings, and transplanting; 2 to 3 months after transplanting and surviving, conducting test cultivation of variety characteristics; and using a variety after confirming that there is no variation in the variety as a subsequent propagation material.


SEQUENCE LISTING (4 sequences)

SEQ ID NO: 1 - DNA, 23 bp, Artificial Sequence
  5' primer of CarMV primer
cggatagtcttgtcaacatacgg

SEQ ID NO: 2 - DNA, 20 bp, Artificial Sequence
  3' primer of CarMV primer
ccttatcgttgcttgcctgt

SEQ ID NO: 3 - DNA, 20 bp, Artificial Sequence
  5' primer of CNFV primer
aaggtatcatcggcagacag

SEQ ID NO: 4 - DNA, 20 bp, Artificial Sequence
  3' primer of CNFV primer
gaagaatctcgtgaagtggc